(19) United States
(12) Patent Application Publication
(10) Pub. No.: US 2025/0000431 A1
(43) Pub. Date: Jan. 2, 2025

(54) Improved detection of evoked potentials

(71) Applicant: ORANGE, Issy-Les-Moulineaux (FR)

(72) Inventors: Cyril Plapous, Chatillon Cedex (FR); Foued Bouchnak, Chatillon Cedex (FR)

(21) Appl. No.: 18/708,758

(22) PCT Filed: Nov. 7, 2022
(86) PCT No.: PCT/EP2022/081001

§ 371 Date: May 9, 2024

(30) Foreign Application Priority Data
Nov. 9, 2021 (FR) FR2111859

(51) Int. Cl. A61B 5/38; A61B 5/372; A61B 5/378

(52) U.S. Cl. CPC A61B 5/38; A61B 5/372; A61B 5/378

(57) ABSTRACT

Method and apparatus for detecting an evoked potential in a physiological signal of a user, in response to the generation of a sensory stimulation signal played by a human machine interface intended for the user. The sensory stimulation signal is periodic and adjustable in frequency. For a given sensory stimulation signal, the frequency of the given signal is adjusted to a first frequency, and then a latency in the user's reaction between the moment the given signal is generated and the moment the evoked potential is detected when such detection occurs is measured. If the latency is lower than a threshold, information is stored according to which the first frequency is attributable to a sensory stimulation signal. Otherwise, the method is repeated with a second frequency in place of the first frequency, the second frequency being different from the first frequency.

### CROSS-REFERENCE TO RELATED APPLICATIONS

This Application is a Section 371 National Stage Application of International Application No. PCT/EP2022/081001, filed Nov. 7, 2022, and published as WO 2023/083755 A1 on May 19, 2023, not in English, which claims priority to French Patent Application No. 2111859, filed Nov. 9, 2021, the contents of which are hereby incorporated by references in their entireties.

### FIELD OF THE DISCLOSURE

The invention relates to the detection of evoked potentials (visual, auditory, or other), via direct neural interfaces.

### BACKGROUND

An evoked potential is a signal that appears in the electroencephalogram (or EEG hereinafter) signals of a user when this user UT is exposed to sensory stimulation (visual, auditory, etc.). With reference to FIG. 1, a specific device comprises a BCI (“Brain-Computer Interface”) headset which allows measuring EEG signals and finding in these signals the frequency of the stimulation (for example visual in FIG. 1). Thus, a human-machine interface such as screen ECR may have a light source (flashing spot on the screen or an LED type of diode in an embodiment that is an alternative to a screen) which flashes at a frequency fn that is lower than the frequency of visual perception. It has been observed that the brain of user UT emits waves having the same frequency as frequency fn of the stimulation source. This frequency can be precisely measured in the EEG signals captured by the BCI headset. This approach is called “SSEVP”, for “Steady-State Evoked Visual Potential”.

An example of a stimulus that is widely used for an SSEVP application is an image (or an area of an image) flashing at a fixed frequency. This stimulation (the flashing) generates a visual evoked potential which consists of a signal of the same frequency as that of the flashing (plus any harmonics). It is possible to measure this frequency in the EEG signals of the person exposed to the flashing. However, user UT must concentrate on this flashing for a long time (typically a few seconds, more particularly between 2 and 4 seconds for example) to ensure that the identified signal does not correspond to an artifact. In addition, EEG signals vary greatly from one person to another and vary widely according to many parameters (fatigue, context, concentration, etc.).

Certain frequencies may therefore be more easily detectable in some people than others. This can also vary within the same person over time and under certain conditions (emotional state, external disruptions, etc.).

These adverse conditions mean that applications using SSVEPs can be relatively slow to respond. For example, it may be necessary to look at a flashing image for several seconds (around 4 seconds or even more) before detecting the evoked potential and succeeding in performing, for example, an action associated with this flashing. If the action consists, for example, of changing the television channel, this manipulation then becomes tedious.

### SUMMARY

An exemplary aspect of the present disclosure relates to a method for detecting an evoked potential in a physiological signal from a user, in reaction to the generation of a sensory stimulation signal reproduced by a human-machine interface intended for the user. The sensory stimulation signal is periodic and adjustable in frequency. In particular, the detection method comprises a selecting of frequencies according to the reaction speed of the user to the sensory simulation signals.

Here, “reproduced by a human-machine interface” is understood to mean having a signal (visual, audio, or other) emitted on said human-machine interface by producing, for example, a periodic flashing on a screen (acting as a human-machine interface) or by playing a periodic audio signal on one or more loudspeakers.

Thus, the implementation of the method makes it possible to modify a frequency of the stimulation signal if said signal does not evoke a reaction in the user's brain after a given latency threshold.

In such an embodiment, for a sensory stimulation signal, the frequency is selected when a reaction latency of the user, between the moment the signal is generated and the moment the evoked potential is detected when such detection occurs, is below a threshold.

This reaction is generally expected after a duration of about 2 to 4 seconds. If it does not occur (for example after 4 seconds), the frequency can be changed to stimulate the user with another frequency.

In such an embodiment, for a sensory stimulation signal, the detection method may comprise:
adjusting the frequency of the signal to a first frequency,
then measuring a reaction latency of the user between the moment the signal is generated and the moment the evoked potential is detected when such detection occurs, and:
if the latency is below a threshold, selecting the first frequency, which comprises a storing of information according to which the first frequency is attributable to a sensory stimulation signal,
otherwise, repeating the method for detecting an evoked potential with a second frequency in place of the first frequency, the second frequency being different from the first frequency.

This second frequency may be lower, to allow the user to concentrate more easily on the stimulation, or conversely may be higher in order to draw their attention. Thus, in general, said second frequency may be higher or lower than the first frequency.

If the first frequency (or the second frequency which replaces the first frequency) has caused a reaction after a duration that is below said threshold, the detection method comprises, if said latency is below the threshold, a storing of information according to which this first frequency is attributed to this given sensory stimulation signal.

In relation to a given sensory stimulation signal, once a frequency has been determined to cause a reaction in the brain after a latency below said threshold, that frequency can be specifically attributed to the given signal. Thus, if said latency is below the threshold, information is stored according to which the first frequency is attributed to this given sensory stimulation signal. By this implementation, with each playing of this stimulation signal by the human-machine interface, the frequency of this signal is such that it causes a reaction whose latency is below said threshold. This implementation can be advantageous for example in the case of displaying flashing targets on a screen at different respective frequencies, and at different respective locations. In this case, if one of the targets resulted in rapid detection at a given location, then if this target is to be displayed again in a subsequent use, it can be displayed for example at the same location on the screen and flash at the same frequency.

Alternatively, in a simple manner, only the frequency that resulted in rapid detection is retained and stored in memory as a usable frequency among other frequencies that are usable for formulating stimulation signals for that user.

For example, respective frequencies may be attributed to a plurality of given sensory stimulation signals, these being frequencies:
chosen from a set of candidate frequencies, and
for which the generated sensory stimulation signal results in detection of an evoked potential after a latency that is below said threshold.

This threshold may be fixed, for example 3 or 4 seconds. Alternatively, it may be relative, as explained in one embodiment below.

Indeed, this threshold may be relative, and the method may comprise, for example:
testing the frequencies of a set of N candidate frequencies in order to identify the K smallest reaction latencies to sensory stimulation signals respectively generated with the N candidate frequencies, with K<N, and
storing information according to which the K frequencies having caused the K smallest reaction latencies are attributable to a sensory stimulation signal.

Thus, these K frequencies are selected independently of a fixed threshold value. For example, the last of the K selected frequencies may be such that their latencies are greater than 3 seconds, while the first of the K selected frequencies may be such that their latencies are less than 3 seconds.

In such an embodiment, said relative threshold may thus correspond to the maximum latency among the K smallest latencies, measured from N generations of sensory stimulation signals using the N respective frequencies of the set of candidate frequencies.

Said method may be implemented during a calibration phase of a device for detecting evoked potential in the physiological signal of a given user, the device being intended for this given user. This may be a calibration of a device specific to the user. Alternatively, this may be a general calibration for a factory setting, with some frequencies being less effective than others overall.

Additionally or alternatively, the method may be implemented during a use, by a given user, of a device for detecting evoked potential in the physiological signal of this given user. In such an embodiment, the selections of the best frequencies are made as the device is used. Typically, when stimulation at a given frequency is not detected or is detected too late, this frequency can be excluded from the set of attributable frequencies, and replaced by a new frequency.

Such an implementation is advantageous in particular for varying the frequencies used according to different moments of the day requiring different attention spans (in the morning or after a meal, for example).

Thus, additionally or alternatively, the method may be implemented at different moments during a day, and it is provided that at least one frequency attributable to a sensory stimulation signal is stored, corresponding to a given moment during a day (moment when latency below a threshold had been measured).

These moments may be particular to a given user. Each moment may in fact be specific to a user. For example, some users are sensitive to certain frequencies in the evening, while others are more sensitive to them in the morning. Thus, the method may be implemented at different moments during a day for this given user, and it is possible to provide for storing at least one frequency attributable to a sensory stimulation signal intended for this given user, with a corresponding given moment during a day. The stored frequency may also be stored with a corresponding identifier of the given user if this user is not the only one using the device.

Furthermore, “storing a frequency” as used above is of course understood to mean storing a frequency index or storing a value of this frequency.

The invention also relates to a computer program comprising instructions for implementing the above method, when these instructions are executed by a processor of a processing circuit.

It also relates to a non-transitory computer medium, storing instructions for a computer program of the above type and readable by a processing circuit in order to execute the above method.

The invention also relates to a device for detecting evoked potential in a physiological signal from a user, comprising:
a generator of sensory stimulation signals, the generator of sensory stimulation signals being connected to a human-machine interface adapted for reproducing the sensory stimulation signal generated for the user, the sensory stimulation signal being periodic and adjustable in frequency,
a frequency selector according to the user's reaction speed to sensory simulation signals.

For example, the device for detecting evoked potential may comprise at least:
a processing circuit configured for (selecting frequencies for which the reaction latencies are lower than said threshold, and) generating (with a frequency thus selected) the sensory stimulation signal,
an output interface connected to the processing circuit on the one hand and to a human-machine interface intended for the user on the other hand, for reproducing the sensory stimulation signal, and
an input interface connected to the processing circuit, for receiving said physiological signal from the user.

For example, the processing circuit may comprise a memory storing at least some identifiers (or values) of respective frequencies of sensory stimulation signals, and information specific to a portion of said frequencies and according to which the frequencies of said portion are commonly attributable to sensory stimulation signals (as a function of the current user and/or as a function of the moment during the current day, or others).

### BRIEF DESCRIPTION OF THE DRAWINGS

Other features and advantages of the invention will become apparent upon reading the description of some exemplary embodiments presented below and upon examining the appended drawings in which:

FIG. 1 shows a system comprising a device for implementing the method of the invention, according to one exemplary embodiment.

FIG. 2 illustrates a method according to one exemplary embodiment.

### DETAILED DESCRIPTION OF ILLUSTRATIVE EMBODIMENTS

It is proposed to select the best flashing frequencies automatically in order to maximize the power of the evoked potential generated. The aim of this automatic selection is to adapt the frequencies to different users and/or to different conditions of use (emotional state, fatigue, external disruptions). Usually the signal used consists of an image with a fixed flashing frequency. This image, when viewed by a human, generates a visual evoked potential of a duration equivalent to the stimulation and which can be detected in the EEG signals recorded via a BCI headset.

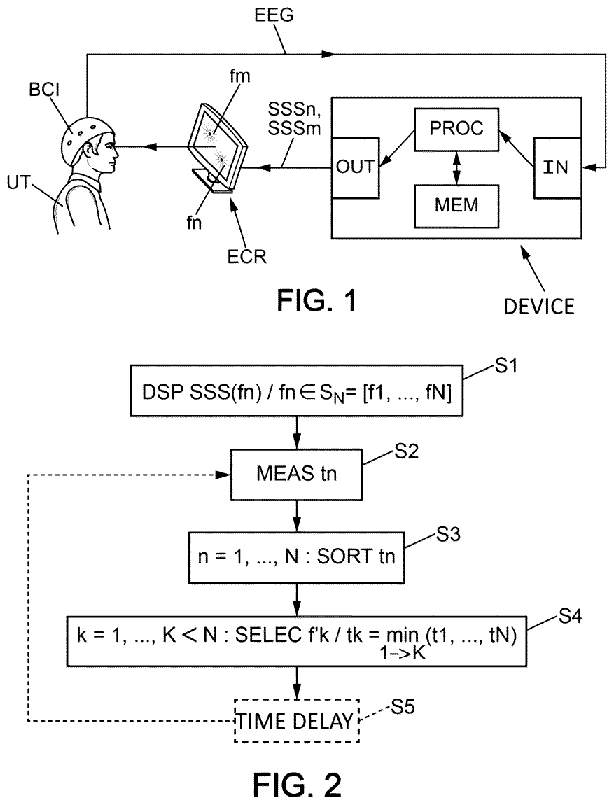

In the following, we consider an application with a human-machine interface for example which requires K frequencies allowing K actions to be carried out. User UT may see for example on screen ECR in FIG. 1 several patterns flashing at different frequencies and each designating a different action (an icon for increasing or decreasing the volume of a television, an icon for changing channels, etc.). User UT focuses his or her gaze on one of the icons flashing at a given frequency fn and the BCI headset identifies frequency fn in the EEG signal. The action corresponding to this frequency can thus be executed. Such a method can make it possible to develop a human-machine interface for people with motor disabilities for example.

A conventional SSVEP solution would then use only K frequencies, which would be sufficient to carry out K actions (a lower number of frequencies may be possible, depending on the applications, because not all the frequencies are necessarily used simultaneously, which in reality would have no impact on the advantages of the invention).

In one embodiment of the present invention, detailed below, it is proposed to use N frequencies with (strictly) N>K, and therefore more frequencies than possible actions.

Usually, a calibration phase is carried out for each user in order to provide robust detection of their evoked potentials. This calibration may be carried out before each use, the aim being to improve the robustness of the detection. Here, the calibration step may be carried out with N frequencies instead of the usual K. At the end of this calibration, it is then possible to have N frequencies available to carry out K actions.

The goal here is to select the best frequencies, meaning those which allow actions to be carried out with the fastest response from the user. Indeed, some frequencies are easier to detect in some people than in others. Furthermore, certain frequencies may also be better suited for certain moments during the day or for certain conditions in a same user (concentration (noise or demands around the user), emotional state, fatigue, or others).

Thus, when initializing the usage session, K frequencies are selected from the N available frequencies. This selection may or may not be random. The system is then used by the user in the usual manner with the ability to carry out K possible actions.

Following the selection of an action (via a flashing frequency in the example of FIG. 1), the system may store in memory the frequency fn which was recognized and how long (delay tn) it took to detect this frequency. Delay tn therefore represents the time difference between the start of the sensory stimulation and the start of the detection of the evoked potential in the user's EEG signal.

For example, the delay tn required to detect this frequency may be calculated after the fact by analyzing the previously recorded EEG signal. The system can analyze the sequence recorded before the moment of detection. Knowing the frequency that was recognized, it is therefore possible to analyze the energy in this frequency band and thus determine how long it had been present before detection.

Subsequently, in order to perform this same action again, a different flashing frequency is associated with it. The choice of this frequency may or may not be random and this frequency must be chosen among those not in use for the time being (namely among (N−K) frequencies for this new step).

Thus, when the user focuses his or her attention in order to select a second action (which may be identical to or different from the first action, although the flashing frequency is strictly different), a frequency different from that of the previous step can be detected in the user's evoked potential.

In the same manner, this new frequency fm as well as its detection delay tm are recorded.

By proceeding in the same manner for the detection of subsequent frequencies, a detection delay ti associated with each of the N available frequencies fi is obtained at the end of step S2 of FIG. 2. It should be noted that the N frequencies tested on the user may be chosen in step S1 of FIG. 2 according to various criteria. A first criterion is not to retain frequencies that are multiples of each other in the set of N frequencies. Indeed, the EEG signal collects frequency fn and possibly the harmonics of this frequency. It is therefore appropriate to exclude multiples of this frequency fn in the set of N frequencies. For example, frequencies such as 10.1 Hz and 10.2 Hz can be retained, as it has been demonstrated that an individual's brain can distinguish a frequency deviation as small as 0.1 Hz and can deliver evoked potentials at these respective frequencies. Interfering frequencies such as the frequencies of the power grid (50 Hz in France) may also be filtered out, and its harmonics or its divisions for example division by two (25 Hz, 12.5 Hz, etc.).

After obtaining a delay tn measured by frequency fn, it is possible to classify these N frequencies in step S3. Indeed, the best frequency is the one enabling the fastest detection (i.e. the shortest delay tn). After having classified these N frequencies fn by order of increasing durations tn, it is possible to select in step S4 the K best frequencies (those whose K respective durations are the smallest) and to allocate them to the K actions used by the system. This allocation may or may not be carried out randomly. The non-random case corresponds to the one where a frequency is chosen for a specific action, for example the frequency best detected is the one whose action is generally used the most (such as “cancel and go back”).

In FIG. 2, the K selected frequencies are denoted f′1, . . . , f′K, the notation “f′” for these frequencies being justified because they are not necessarily identical to the first K frequencies in the set Sn of N frequencies f1, . . . , fK, . . . , fN.

Such an implementation therefore makes it possible to automatically optimize the selection of frequencies for each user. In the same manner, as soon as the conditions of use vary with time and with the moment during a day (user attention differs after a meal compared to the morning for example), it is possible to program the system to adapt to these different moments by selecting the frequencies giving the best performance in terms of delay in detecting the evoked potential.

In the case where the system is used continuously or over a long period of time, external conditions or the mental state of the user may vary. A previously discarded frequency (among N frequencies) may now be better than the K frequencies currently in use. It is therefore of interest to allow for the possibility of reintroducing the discarded frequencies by repeating steps S2 to S4 for a new selection of K frequencies. This may be carried out after a time delay (step S5 in dotted lines) that was previously set (for example after a few hours of use) or via a voluntary action by the user, for example.

Thus, the invention combines the following advantages:
Automatic selection of K frequencies giving the best performance in terms of response time;
Automatic adaptation of the system to different possible users;
Robustness of the system to changes in the conditions of use (emotional state, fatigue, external disruptions).

The invention finds numerous applications, in particular all applications using SSVEP (while improving this approach). It may, for example, allow controlling a smart home via a BCI headset (turn on the television, change channels, turn on lights, turn off the furnace, etc.)

Of course, the invention is not limited to the embodiment presented above. It extends to other variants.

For example, a calibration phase was described above as the first step in selecting K frequencies among the N possible frequencies. However, during calibration, a certain number of elements may be stored, in particular the power in the frequency band of interest and the duration of the period of time during which this power is observed. Then, when the user uses the system, the N frequencies are each used once and these frequencies are detected due to the elements (power, duration, etc.) stored during the calibration phase. Then detection delay tn, measured for each frequency fn, makes it possible to classify the frequencies from best to worst (based on the criterion of detection delay).

This classification of frequencies may be carried out during the calibration phase itself as described above, or simply throughout use, typically by detecting the reaction time at each frequency used for an action for example.

Furthermore, a reaction to visual stimulation (typically on a screen ECR) was described above. However, reactions to auditory stimulation in particular have also been observed. One or more loudspeakers may play sounds (for example beeps) of different frequencies and a user focusing on one of the sounds can thus select an action.

An exemplary aspect of the present disclosure improves the situation of the prior art.

Although the present disclosure has been described with reference to one or more examples, workers skilled in the art will recognize that changes may be made in form and detail without departing from the scope of the disclosure and/or the appended claims.

1. A detection method implemented by a device and comprising:
generating sensory stimulation signals to be applied to a user by a human machine interface intended for the user, the sensory stimulation signals being periodic and adjustable in frequency;
detecting evoked potentials in physiological signals of the user, as respective reactions to the generating of the sensory stimulation signals; and
selecting frequencies of the sensory stimulation signals according to a reaction speed of the user to the sensory simulation signals.

2. The detection method according to claim 1, wherein, for a particular sensory stimulation signal of the sensory stimulation signals, the frequency is selected when a reaction latency of the user, between a moment the signal is generated and a moment the evoked potential is detected, when such detection occurs, is below a threshold.

3. The detection method according to claim 1, wherein, for a particular sensory stimulation signal of the sensory stimulation signals, the detection method comprises:
adjusting the frequency of the signal to a first frequency,
then measuring a reaction latency of the user between the moment the signal is generated and the moment the evoked potential is detected, when such detection occurs,
and:
if the latency is below a threshold, selecting the first frequency, which comprises a storing of information according to which the first frequency is attributable to the sensory stimulation signal,
otherwise, repeating the generating and detecting with a second frequency in place of the first frequency, the second frequency being different from the first frequency.

4. the detection method according to claim 3, wherein the detection method comprises, if said latency is below the threshold, storing information according to which the first frequency is attributed to said particular sensory stimulation signal.

5. The method according to claim 2, wherein respective frequencies are attributed to the sensory stimulation signals, these being frequencies:
chosen from a set of candidate frequencies, and
for which the generated sensory stimulation signal results in detection of an evoked potential after a latency that is below said threshold.

6. The detection according to claim 2, wherein said threshold is relative, and the method comprises:
testing frequencies of a set of N candidate frequencies in order to identify smallest reaction latencies to sensory stimulation signals respectively generated with the N candidate frequencies, with K<N, and
storing information according to which the K frequencies having caused the K smallest reaction latencies are attributable to respective sensory stimulation signals.

7. The detection method according to claim 1, implemented during a calibration phase of the device, the device being intended for this given user.

8. The detection method according to claim 1, implemented during a use, by the user, of the device of this user.

9. The detection method according to claim 1, implemented at different moments during a day and comprising storing at least one frequency attributable to a respective sensory stimulation signal of the sensory stimulation signals, corresponding to a respective moment during that day.

10. The detection method according to claim 8, implemented at different moments during a day, for the user, and comprising storing at least one frequency attributable to a respective sensory stimulation signal of the sensory stimulation signals intended for that user, corresponding to a respective moment during that day.

11. A non-transitory computer readable medium comprising instructions stored thereon for implementing a method for detecting evoked potentials in a physiological signals of the user, when said instructions are executed by a processor of a processing circuit, wherein the method comprises:
generating sensory stimulation signals to be applied by a human machine interface intended for the user, the sensory stimulation signals being periodic and adjustable in frequency;
detecting the evoked potentials in the physiological signals of the user, in reaction to the generating of the sensory stimulation signals; and
selecting frequencies of the sensory stimulation signals according to a reaction speed of the user to the sensory simulation signals.

12. A device for detecting an evoked potential in a physiological signal from a user comprising:
a generator of sensory stimulation signals, the generator of sensory stimulation signals being connectable to a human-machine interface adapted for reproducing the sensory stimulation signals generated for the user, the sensory stimulation signals being periodic and adjustable in frequency, and
a frequency selector for the sensory stimulation signals according to the user's reaction speed to the sensory stimulation signals.

13. The device for detecting the evoked potential according to claim 12, wherein the detection device comprises at least:
a processing circuit configured for generating the sensory stimulation signals and selecting at least one frequency,
an output interface connected to the processing circuit and connectable to the human-machine interface intended for the user, and
an input interface connected to the processing circuit, for receiving said physiological signal from the user.

14. The device according to claim 13, wherein the processing circuit comprises a memory storing at least some identifiers of respective frequencies of the sensory stimulation signals, and information specific to a portion of said frequencies and according to which the frequencies of said portion are commonly attributable to the sensory stimulation signals.
